(19) United States
(12) Patent Application Publication
(10) Pub. No.: US 2012/0035527 A1
(43) Pub. Date: Feb. 9, 2012

(54) PHOTOCHEMICAL TISSUE BONDING

(75) Inventors: Guillermo Tearney, Cambridge, MA (US); Brett Bouma, Quincy, MA (US); Robert W. Redmond, Newton Centre, MA (US); Irene E. Kochevar, Charlestown, MA (US)

(73) Assignee: THE GENERAL HOSPITAL CORPORATION, Boston, MA (US)

(21) Appl. No.: 12/681,313

(22) PCT Filed: Oct. 2, 2008
(86) PCT No.: PCT/US08/78639

§ 371 Date: Oct. 21, 2011

Related U.S. Application Data
(60) Provisional application No. 60/997,472, filed on Oct. 3, 2007.

(51) Int. Cl. A61B 18/18

(52) U.S. Cl. 604/20; 606/8

(57) ABSTRACT

The exemplary embodiments of the present invention provides exemplary methods for adhering biological membranes to luminal anatomical structures using photosensitizing agents and electromagnetic energy. The exemplary embodiments of the present invention also provides method for stabilizing luminal anatomical structures. Further, exemplary embodiments of the present invention provide arrangements for coupling to a luminal anatomical structure.

### RELATED APPLICATIONS/PATENTS AND INCORPORATION BY REFERENCE

This International application claims priority to U.S. Provisional Application No. 60/997,472, filed Oct. 3, 2007, the entire contents of which are incorporated herein by reference.

The foregoing applications, and all documents cited therein and all documents cited or referenced therein, and all documents cited or referenced herein, including any U.S. or foreign patents or published patent applications, International patent applications, as well as, any non-patent literature references and any manufacturer's instructions, are hereby expressly incorporated herein by reference.

### GOVERNMENT SUPPORT

Research supporting this application was supported by Department of Defense, Medical Free Electron Problem, Contract No. FA9550-04-1-0079. The government may have certain rights in the invention.

### FIELD OF THE INVENTION

The present invention relates to a field of photochemical tissue bonding, and in particular to methods for adhering a biological membrane to a luminal anatomical structure and arrangements for coupling to the luminal anatomical structure.

### BACKGROUND

Stents are devices for maintaining patency of anatomic luminal structures, typically fabricated from metals, such as nitinol, composite, plastic, or other solid non-biological material and expanded intraluminally. Stents may be patterned so that the stent struts allow re-endothelialization or re-epithelialization of the stent surface. In addition stent struts may be structured so that they allow passage of intraluminal tissue such as blood, bile, or lymph, through said strut openings. Stent struts may be coated with peptides or drug agents, such as antibiotic agents, chemotherapeutic agents to prevent an overly aggressive host response to the stent such as restenosis or to encourage re-endothelialization of the stent strut surface.

Stents can be deployed using a stent deployment platform, which typically comprises a balloon which when inflated embeds the stent within the luminal anatomical structure surface. Stents may have mechanical characteristics that allow it to “self-expand”, thereby not necessarily relying on the balloon deployment mechanism to place the stent strut at the surface of the luminal anatomic structure. Structural and mechanical characteristics of the stent allow it to be inserted into the luminal anatomic structure and deployed such that when deployed, the structural and mechanical characteristics of the stent maintain at least partial patency of the said luminal anatomic structure. Stents may additionally contain structural and mechanical portions, commonly at the proximal and distal ends, that prevent longitudinal movement of the stent within the luminal anatomic structure.

Stent devices as described above may have undesirable characteristics. For example, even though the stents are typically comprised of inert materials, they are not commonly biological materials and the host responds to the foreign material unnaturally. In addition, mechanical characteristics of the stent, including their radial compressibility, may be needed to maintain patency of the stent and luminal anatomic structure. In order for these mechanical characteristics to be realized, the stent usually needs to be deployed in a manner that damages the luminal structure. Damage to the luminal anatomic structure causes an unnatural healing response to the stent, which can result in subsequent closing or restenosis of the luminal structure or improper re-endothelialization of epithelialization of the stent surface, which impairs the natural biological function of the luminal anatomic structure. It is therefore desirable to have a device for maintaining patency where the device is comprised of a biological material, can impart mechanical stability, but does not require destructive deployment.

Additionally, owing to the mechanical properties of the stent, the stent should usually comprise a circular cylindrical topology, which can require coverage of areas of the anatomical structure that may not need covering, thereby increasing the risk of adverse consequences. Furthermore, this required circular cylindrical topology may not be appropriate if the luminal structure does not share the diameter or same circularly cylindrical topology as the stent. This topology mismatch can occur frequently and malapposition of the stent struts results. Malapposition further causes the host to respond to the stent in an unnatural manner, further increasing the likelihood of future adverse complications. It may therefore be desirable to have a deformable device that at least partially conforms to the topology of the luminal anatomic structure.

For most stent applications, the purpose of the stent device may be to keep open and maintain patency of a luminal anatomic structure that has a collapsed, occluded, or at least partially compromised lumen. Stent devices therefore generally must be capable of maintaining patency of a luminal anatomic structure. There are circumstances in medicine however, when maintaining luminal patency is not the primary function of the stent. One such example is the stabilization of certain types of atherosclerotic plaques that are termed thin-capped fibroatheromas (TCFA). Thin-capped fibroatheroma is a histopathologic definition of an atherosclerotic plaque that comprises a thin fibrous or collagen-containing cap overlying a lipid-rich area containing necrotic debris. TCFA have been implicated as the culprit lesion that gives rise to the majority of acute myocardial infarctions, acute coronary syndromes, and sudden cardiac death. It is thought that inflammatory cells present within or near the cap degrade the mechanical integrity of the cap by producing proteinases including collagenases such as metalloproteinases and cathepsins. As a result, extrinsic or intrinsic mechanical forces can cause the cap to break or rupture, exposing the thrombogenic lipid to blood. When this event occurs, a thrombus may form at the site of rupture, partially or totally occluding the lumen of the vessel, causing acute coronary syndrome or acute myocardial infarction, respectively.

Current stenting platforms have been provided to prospectively treat these types of atherosclerotic lesions to prevent subsequent rupture. However, the nature of common stent deployment mechanisms is destructive of the luminal structure, thereby causing rupture upon deployment. This iatrogenic rupture causes emboli to be sent downstream, which lodge in arteries, further causing infarction. Later, malapposition and tissue alteration causes unnatural biological response to the stent that can result in improper endothelialization of the stent and restenosis. Current stents also cause difficulties when inserted into the artery owing to the thrombogenic nature of the stent strut surface that may cause a thrombus to form, resulting in occlusion of the lumen.

Thus, there may be a need in the field for a method to place a stabilizing device over the susceptible areas of the artery wall that contain TCFA that do not disrupt the luminal anatomic structure and that provide good apposition to the arterial wall. For this application it is also desirable that the device allow proper blood flow after deployment without causing a thrombus.

Accordingly, there may be a need to overcome the deficiencies as described herein above.

### SUMMARY OF THE INVENTION

Certain exemplary embodiments of the instant invention can provide an exemplary use of photoactivatable biological membrane (PAM) as a luminal covering material. Other exemplary embodiments of the instant invention can provide exemplary methods for covering, repairing, and maintaining the patency of an anatomic structure, for example a luminal anatomic structure, using a photoactivatable biological membrane.

Accordingly, in one aspect, the invention provides for a method for adhering a biological membrane to a luminal anatomical structure, the method comprising: contacting a biological membrane with a photosensitizer agent; deploying the biological membrane-photosensitizer complex to the luminal anatomical structure of interest; and applying electromagnetic energy; thereby adhering the biological membrane to the luminal anatomical structure.

A further embodiment of the present invention relates to a method for stabilizing a luminal anatomical structure, the method comprising: contacting a biological membrane with a photosensitizer agent; deploying the biological membrane to the luminal anatomical structure in need of stabilization; applying electromagnetic energy to the biological membrane-photosensitizer complex in a manner effective to bond the tissue; thereby stabilizing a luminal anatomical structure.

Still further another embodiment of the present invention relates to a method for treating or preventing an atherosclerotic plaque, the method comprising: identifying an atherosclerotic plaque; contacting a biological membrane with a photosensitizer agent; deploying the biological membrane to the atherosclerotic plaque; applying electromagnetic energy to the biological membrane-photosensitizer complex in a manner effective to bond the tissue; thereby treating or preventing an atherosclerotic plaque.

The electromagnetic energy in these methods can be applied to the one or both of the biological membrane and the luminal anatomical structure, also in a manner effective to bond the tissue.

The electromagnetic energy can be electromagnetic radiation, particularly at a wavelength between about 532 nm and 660 nm.

In the methods of the present invention, the contacting step can occur ex vivo or in vivo in a subject.

Another aspect of the present invention relates to a photoactivatable membrane device comprising a biological membrane and a photosensitizer agent.

The biological membrane of the present methods can be an amniotic membrane from a mammal and, in particular, from a human. The biological membrane can be synthetic or natural and can contain an epithelial layer, a basement membrane layer, and a connective tissue layer.

In a further embodiment of the present invention, the photosensitizer agent is coated on the biological membrane. The photosensitizer agent can be coated on one or more of the epithelial layer, the basement membrane layer or the connective tissue layer of the biological membrane and is coated on the biological membrane in a gradient from highest concentration to lowest concentration.

The biological membrane can be coated with biological molecules such as anticoagulants, molecules that enhance cell migration, proteases, molecules that enhance chemotaxis, chemoattractants, immunosuppressive agents, chemotherapeutic agents, molecules that enhance cell growth, molecules that enhance healing, antibodies, small molecule inhibitors, anti-inflammatory agents and antioxidants.

The photosensitizer agent can be a compound selected from the group consisting of: xanthenes, flavins, thiazines, porphyrins, expanded porphyrins, and chlorophylis, wherein the photosensitizer agent is activated using electromagnetic energy, in particular, electromagnetic radiation, and more particularly, using electromagnetic radiation which is centered at a wavelength between about 532 nm and 660 nm.

Further still, the present invention relates to a method for promoting cell growth in a luminal anatomical structure of interest, the method comprising: contacting a biological membrane with a photosensitizer agent; deploying the biological membrane-photosensitizer complex to the luminal anatomical structure of interest; and applying electromagnetic energy; thereby promoting cell growth and migration in a luminal anatomical structure of interest.

The method for promoting cell growth in a luminal anatomical structure of interest can be a cell type of at least one of epithelial or endothelial cell and wherein the cell growth mitigates thrombosis.

In addition, in the method of adhering a biological membrane to a luminal anatomical structure or the method for promoting cell growth in a luminal anatomical structure of interest the biological membrane is placed within a lumen of a luminal anatomic structure. The luminal anatomic structure is not altered prior to performing the method. Alternatively, the epithelial cells or endothelial cells of the luminal anatomic structure are removed prior to performing the method.

In another embodiment of a method of adhering a biological membrane to a luminal anatomical structure or the method for promoting cell growth in a luminal anatomical structure of interest the luminal anatomic structure is pretreated prior to performing the method. The pretreatment of the luminal anatomic structure is selected from methods consisting of abrasion or drying.

In still another embodiment of a method of adhering a biological membrane to a luminal anatomical structure or the method for promoting cell growth in a luminal anatomical structure of interest, the biological membrane is comprised of one or more layers of membrane sheets and the layers of membrane sheets can be affixed to one another by electromagnetic radiation.

In an additional embodiment of a method of adhering a biological membrane to a luminal anatomical structure or the method for promoting cell growth in a luminal anatomical structure of interest, the biological membrane is conformed in a geometry which can be a cylinder, a plane, any geometry preformed to conform to the contour of the tissue of interest, or preformed to conform any desired contour. For example, the cylinder can be used as a stent or covering and the biological membrane can have one or more holes wherein the holes can be between 10-750 μm in diameter.

In one other embodiment of a method of adhering a biological membrane to a luminal anatomical structure or the method for promoting cell growth in a luminal anatomical structure of interest the edges of the biological membrane are tapered and the edges of the biological membrane can contain projections. These projections comprise at least one of a biological membrane, amniotic membrane, metal struts, nitinol struts, plastic struts, or composites, such as Polytetrafluoroethylene (PTFE), Teflon, plastic, rubber, nitinol, or biodegradable composites or the like.

In still a further embodiment of a method of adhering a biological membrane to a luminal anatomical structure or a method for promoting cell growth in a luminal anatomical structure of interest there can be a further step of applying a second dose of electromagnetic energy is performed and the electromagnetic energy can be applied before or after deployment of the biological membrane. Also, the electromagnetic energy can be applied after bonding of the biological membrane to the anatomical structure and the electromagnetic energy is electromagnetic radiation which can be centered at a wavelength between about 532 nm and 660 nm.

Also, the present invention relates to a kit comprising a biological membrane and a photosensitizer agent for use in the methods of the present invention, and instructions for use.

The present invention also relates to an arrangement for coupling to a luminal anatomical structure, comprising a tissue structure having a form so as to be transportable within the luminal anatomical structure, wherein the tissue structure includes a photo-activatable substance which, when activated by at least one electro-magnetic radiation, couples to at least one portion of the luminal anatomical structure.

The luminal anatomical structure can be a coronary artery or a membrane. In addition, the form of the tissue structure can be a cylinder or a stent and each of which can have at least one hole, layers of membranes, tapered ends or anchored ends.

The invention relating to an arrangement for coupling to a luminal anatomical structure can also include a step where the tissue structure is irradiated using the at least one electro-magnetic radiation at least one of prior to, during or after an insertion thereof into the luminal anatomical structure. The irradiation of the tissue structure can cause at least one change of at least one mechanical characteristic of the tissue structure.

The tissue structure further can include at least one additional substance which is configured to modify at least one of clotting, healing, immunity or cell growth of the luminal anatomical structure in response to a coupling of the tissue structure to the luminal anatomical structure.

In addition, prior to an application of the tissue structure to the luminal anatomical structure, the tissue structure is modified to improve healing of the luminal anatomical structure.

The invention relating to an arrangement for coupling to a luminal anatomical structure can further comprise an expandable apparatus which is coupled to the tissue structure prior to application of the tissue structure to the luminal anatomical structure. The expandable apparatus can be a balloon which, when expanded, deposits the tissue structure unto the luminal anatomical structure. Further, the expandable apparatus can be a compliant low-pressure balloon which, when expanded, is restricted from damaging the luminal anatomical structure.

The invention relating to an arrangement for coupling to a luminal anatomical structure can further comprise an optical fiber apparatus which is configured to provided the at least one electro-magnetic radiation.

Still further, the invention relating to an arrangement for coupling to a luminal anatomical structure can comprise a guidance apparatus which is configured to provided a location of application of the tissue structure on the luminal anatomical structure.

These and other objects and embodiments are described in or are obvious from and within the scope of the invention, from the following Detailed Description.

### BRIEF DESCRIPTION OF THE DRAWINGS

Further objects, features and advantages of the invention will become apparent from the following detailed description taken in conjunction with the accompanying figures showing illustrative embodiments of the invention, in which:

FIG. 1 is a diagram of at least one portion of an exemplary embodiment of a photoactivatable biological membrane (PAM) device;

FIG. 2 (a-c) are diagrams of different exemplary PAM device configurations or topologies (FIG. 2a depicts an exemplary PAM device configuration that comprises a sheet of PAM, FIG. 2b depicts a, exemplary PAM device configuration whereby the PAM device is a circular cylinder, and FIG. 2c shows an exemplary PAM device configuration whereby the PAM device is an overlapping circular cylinder);

FIG. 3 (a-c) is a diagram of exemplary PAM device patterns (FIG. 3a shows an exemplary PAM device with holes, FIG. 3b shows an exemplary PAM device pattern where the edges of the PAM device are tapered to further significantly improve endothelial or epithelial cell migration, and FIG. 3c depicts an exemplary PAM device where the PAM device configuration is comprised of layers of amniotic membrane configured to impart substantially more thickness and/or mechanical stability to the PAM device);

FIG. 4 (a and b) is a diagram of exemplary PAM device anchoring patterns, where FIG. 4a is a coronal view of an exemplary PAM device, and FIG. 4b is a cross-sectional view of the exemplary PAM device;

FIG. 5 is a diagram of an exemplary PAM device with coatings;

FIG. 6 is a diagram of an exemplary PAM system;

FIG. 7 (a-c) are diagrams of an exemplary balloon PAM device deployment device catheter (FIG. 7a is a diagram of an exemplary embodiment of the balloon PAM deployment device prior to exposing the balloon to the lumen of the luminal anatomic structure, FIG. 7b is a diagram of the exemplary balloon PAM deployment device after exposing the balloon to the lumen of the luminal anatomic structure, and FIG. 7c is a diagram of the exemplary PAM device balloon deployment device after the balloon is inflated, placing the PAM device in substantially close proximity to the luminal surface of the luminal anatomic structure);

FIG. 8 (a and b) are exemplary diagrams of optical arrangements within the PAM device deployment device catheter, where FIG. 8a is a diagram of an exemplary embodiment of the optical arrangement of one embodiment of the exemplary PAM device deployment device whereby the electromagnetic radiation is configured to illuminate at least one portion of the exemplary PAM device and luminal surface of the luminal anatomic structure, and FIG. 8b is a diagram of the optical arrangement of one exemplary embodiment of the PAM device deployment device whereby the electromagnetic radiation is configured to diffuse the electromagnetic radiation over a substantial portion of the exemplary PAM device and luminal surface of the luminal anatomic structure);

FIG. 9 is a flow chart depicting an exemplary embodiment of a process of deploying and activating a PAM device in a coronary artery in accordance with the present invention; and

FIG. 10 is a diagram of an exemplary embodiment of a PAM device configured to be associated with a stent.

### DETAILED DESCRIPTION OF EXEMPLARY EMBODIMENTS

### Exemplary Definitions

Unless defined otherwise, all technical and scientific terms used herein have the meaning commonly understood by a person skilled in the art to which this invention belongs. The following references, the entire disclosures of which are incorporated herein by reference, provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms may have the meanings ascribed to them below, unless specified otherwise. However, it should be understood that other meanings that are know or understood by those having ordinary skill in the art are also possible, and within the scope of the present invention.

By “atherosclerotic plaque” can mean, but in no way limited to, a lesion that results from atherosclerosis, or the like. For example, often a plaque is a buildup of lipids, cholesterol, calcium, and cellular debris within the intima of the vessel wall.

By “biological membrane” can mean, but in no way limited to, an organized layer of cells taken from any animal. In preferred embodiments, the biological membrane is an amniotic membrane. In other exemplary embodiments, the biological membrane can be taken from the amnion of a mammal, for example a cow, pig, sheep, or the like. In another preferred embodiment, the biological membrane may be taken from, for example, a human pregnancy, post partum.

By “cell growth” can mean, but in no way limited to, the process of cell division or cell proliferation or the like.

By “electromagnetic energy” can mean, but in no way limited to, electromagnetic radiation, or the like. For example, electromagnetic radiation can include light having a wavelength in the visible range or portion of the electromagnetic spectrum, or in the ultra violet and infrared regions of the spectrum.

By “luminal anatomical structure” can mean, but in no way limited to, a structure that is found on the luminal surface of, for example, a blood vessel or another anatomical conduit.

By “luminal surface” can mean, but in no way limited to, the inner surface. A lumen is an interior space or cavity, for example, the interior of a blood vessel. The luminal surface of a blood vessel is the side facing the blood. For example, the luminal (or apical) side of an epithelial cell is the side that communicates with the lumen of the tube the epithelium lines.

By “photosensitizer agent” can mean, but in no way limited to, a chemical compound that produces a biological effect upon photoactivation or a biological precursor of a compound that produces a biological effect upon photoactivation, or the like. Exemplary photosensitizers can be those that absorb electromagnetic energy. Exemplary photosensitizer agents may include, but are not limited to, Rose Bengal, riboflavin-5-phosphate, and methylene blue.

By “photoactivatable membrane device” can mean, but in no way limited to, a membrane that is capable of photoactivation, or the like. Photoactivation can be used to describe the process by which energy is absorbed by a compound, e.g., a photosensitizer, thus “exciting” the compound, which then becomes capable of converting the energy to another form of energy, preferably chemical energy.

By “thin-capped fibroatheroma (TCFA)” can mean, but in no way limited to, a type of atherosclerotic plaque, or the like. A TCFA can comprise a thin fibrous or collagen-containing cap overlying a lipid-rich area containing necrotic debris.

### Exemplary Photoactivation and Photosensitizers

Photoactivation, as referred to herein, e.g., can be used to describe the process by which energy in the form of electromagnetic radiation is absorbed by a compound, e.g., a photosensitizer, thus “exciting” the compound, which then becomes capable of converting the energy to another form of energy, preferably chemical energy. The electromagnetic radiation can include energy, e.g., light, having a wavelength in the visible range or portion of the electromagnetic spectrum, or the ultra violet and infrared regions of the spectrum. The chemical energy can be in the form of a reactive species, e.g., a reactive oxygen species, e.g., a singlet oxygen, superoxide anion, hydroxyl radical, the excited state of the photosensitizer, photosensitizer free radical or substrate free radical species. The exemplary photoactivation process described herein can involve an insubstantial transfer of the absorbed energy into heat energy. Preferably, photoactivation occurs with a rise in temperature of less than 3 degrees Celsius (° C.), more preferably a rise of less than 2 degrees ° C. and even more preferably, a rise in temperature of less than 1 degree ° C. as measured, e.g., by an imaging thermal camera that looks at the tissue during irradiation. The camera can be focused in the area of original dye deposit, e.g., the wound area, or on an area immediately adjacent the wound area, to which dye will diffuse. As used herein, a “photosensitizer” is a chemical compound that produces a biological effect upon photoactivation or a biological precursor of a compound that produces a biological effect upon photoactivation. Exemplary photosensitizers can be those that absorb electromagnetic energy, such as light. While not wishing to be bound by theory, the photosensitizer may act by producing an excited photosensitizer or derived species that interacts with tissue, e.g., collagenous tissue, to form a bond, e.g., a covalent bond or crosslink. Certain exemplary photosensitizers typically have chemical structures that include multiple conjugated rings that allow for light absorption and photoactivation. A number of photosensitizers are known to one of skill in the art, and generally include a variety of light-sensitive dyes and biological molecules. Examples of photosensitive compounds can include, but are not limited to, xanthenes, e.g., rose bengal and erythrosin; flavins, e.g., riboflavin; thiazines, e.g., methylene blue; porphyrins and expanded porphyrins, e.g., protoporphyrin I through protoporphyrin IX, coproporphyrins, uroporphyrins, mesoporphyrins, hematoporphyrins and sapphyrins; chlorophylis, e.g., bacteriochlorophyll A, and photosensitive derivatives thereof. Exemplary photosensitizers according to the methods of the invention as described herein are compounds capable of causing a photochemical reaction capable of producing a reactive intermediate when exposed to light, and which do not release a substantial amount of heat energy. Some exemplary photosensitizers include Rose Bengal (RB); riboflavin-5-phosphate (R-5-P); methylene blue (MB); and N-hydroxypyridine-2-(1H)-thione (N-HTP).

In certain exemplary embodiments, the photosensitizer agent, e.g., RB, R-5-P, MB, or N-HTP, can be dissolved in a biocompatible buffer or solution, e.g., saline solution, and used at a concentration of from about 0.1 mM to 10 mM, preferably from about 0.5 mM to 5 mM, more preferably from about 1 mM to 3 mM.

The photosensitizer agent can be administered to the luminal surface by the PAM device as described herein. The PAM device may be delivered in any number of ways. RB can be administered in the following exemplary ways. 1. Pre-coating the external surface of the stent with RB prior to positioning. 2. Application of RB to the luminal surface by means of (a) spray (b) applicator (sponge, swab, etc) (c) filling vessel with RB solution and purging after 60 seconds.

The exemplary device may be insertable or the exemplary device may be implantable.

The electromagnetic radiation, e.g., light, can be applied to the tissue at an appropriate wavelength, energy, and duration, to cause the photosensitizer to undergo a reaction to affect the structure of the amino acids in the tissue, e.g., to cross-link a tissue protein, thereby creating a tissue seal. The wavelength of light can be chosen so that it corresponds to or encompasses the absorption of the photosensitizer, and reaches the area of the tissue that has been contacted with the photosensitizer, e.g., penetrates into the region where the photosensitizer is injected. The electromagnetic radiation, e.g., light, necessary to achieve photoactivation of the photosensitizer agent can have a wavelength from about 350 nm to about 800 nm, preferably from about 400 to 700 nm and can be within the visible, infra red or near ultra violet spectra. The energy can be delivered at an irradiance of about between 0.5 and 5 W/cm², preferably between about 1 and 3 W/cm². The duration of irradiation can be sufficient to allow cross-linking of one or more proteins of the tissue, e.g., of a tissue collagen. For example, in corneal tissue, the duration of irradiation can be from about 30 seconds to 30 minutes, preferably from about 1 to 5 minutes. The duration of irradiation can be substantially longer in a tissue where the light has to penetrate a scattering layer to reach the wound, e.g., skin or tendon. For example, the duration of irradiation to deliver the required dose to a skin or tendon wound can be at least between one minute and two hours, preferably between 30 minutes to one hour.

Suitable sources of electromagnetic energy can include but not limited to commercially available lasers, lamps, light emitting diodes, or other sources of electromagnetic radiation. Light radiation can be supplied in the form of a monochromatic laser beam, e.g., an argon laser beam or diode-pumped solid-state laser beam. Light can also be supplied to a non-external surface tissue through an optical fiber device, e.g., the light can be delivered by optical fibers threaded through a small gauge hypodermic needle or an arthroscope. Light can also be transmitted by percutaneous instrumentation using optical fibers or cannulated waveguides.

The choice of energy source can generally be made in conjunction with the choice of photosensitizer employed in the method. For example, an argon laser can be an energy source suitable for use with RB or R-5-P because these dyes are optimally excited at wavelengths corresponding to the wavelength of the radiation emitted by the argon laser.

Other suitable combinations of lasers and photosensitizers are known to those of skill in the art. Tunable dye lasers can also be used with the methods described herein.

### Biological Membranes

The exemplary embodiment of the PAM device according to the instant invention comprises a biological membrane. A biological membrane as used in the method and device of the exemplary embodiment of the present invention described herein can be taken from any mammal. For example, in certain exemplary embodiments, the biological membrane may be taken from the amnion of a mammal, for example a cow, pig, sheep, or the like. In another exemplary embodiment, the biological membrane may be taken from, for example, a human pregnancy, post partum.

The amniotic membrane is the translucent innermost layer of the three layers forming the fetal membranes, and is derived from the fetal ectoderm. The amniotic membrane contributes to homeostasis of the amniotic fluid. At maturity, the amniotic membrane is composed of epithelial cells on a basement membrane, which in turn is connected to a thin connective tissue membrane or mesenchymal layer by filamentous strands.

The isolated amniotic membranes that can be used in the exemplary embodiment of the present invention may be obtained from a commercial source, for example from suppliers such as AmbioDry and AmbioDry2 from OKTO Ophtho and AMNIOGRAFT from Bio-Tissue. Alternatively, the amniotic membrane may be recombinant, or naturally occurring and sterilized. The amniotic tissue may be obtained postpartum and then preserved by any number of methods known to one of skill in the art (e.g. glycerol, lyophilization, gluteraldehyde, etc). Additionally, amniotic membranes that are derived from non-humans may be used.

The membranes of the exemplary embodiment of the present invention can be, for example, between 10 μm, 15 μm, 20 μm, 25 μm, 30 μm, 35 μm or more pm in thickness. In certain exemplary embodiments, the membrane is 20 μm in thickness, and is an amniotic membrane.

### Membrane Devices

Previous research has shown that the physical properties of a membrane, in particular a photoactivatable membrane (PAM), can be altered by photocrosslinking the constitutive proteins. For example, in one example, a tube was prepared by applying rose bengal to a strip of PAM, wrapping 3-4 layers around a rod, irradiating and then removing the rod [New England Society of Plastic and Reconstructive Surgeons. New Castle, N.H. Jun. 2-4, 2006; Photochemical sealing improves outcome following peripheral neuropathapy. O'Neill AC*, Bujold K E, Randolph M A, Kochevar I E, Redmond R W, Winograd J M.], incorporated by reference in its entirety herein. Previous studies have also shown that flat layers of human amniotic membrane can be photocrosslinked together [unpublished], incorporated by reference in its entirety herein.

Further, the amniotic membranes of the exemplary embodiment of the present invention may be modified to change their consistency. For example, amniotic membranes with enhanced rigidity as biocompatible devices are described in WO06002128, incorporated by reference in its entirety herein.

A membrane device may be formed prior to deployment, during, or after deployment, in order to conform and/ or alter the topology of the structure to which it is to be applied. Accordingly, in certain embodiments, the PAM device is designed to alter the topology of a luminal anatomic structure.

In one such example, the membrane device may be formed as a sheet of membrane, for example a sheet of amniotic membrane. This configuration may be preferable for use in imparting stability to one portion of the luminal anatomical structure.

In certain other examples, the intraluminal covering device that attaches to a luminal anatomic structure can at least partially cover the anatomical structure in a manner that either at least partially maintains the patency of said luminal anatomic structure.

In other examples, the membrane, preferably the exemplary PAM, attaches to a luminal anatomic structure that does not move within said structure following deployment. In other preferred examples, the membrane, preferably the PAM, can attach to a luminal anatomic structure that at least partially covers the anatomical structure in a manner that either at least partially stabilizes of said luminal anatomic structure.

It may be preferred that the membrane, for instance the exemplary PAM that attaches to a luminal anatomic structure does not damage said structure.

In another example, this topology may be used to repair a defect in an anatomical structure. In certain cases, it may be preferable to use the membrane of the invention to treat, repair, or cover only one portion of an anatomical structure, and leave the other portion of the anatomical structure intact. For example, to cover only a portion of the luminal anatomic structure that may utilize an alteration while leaving the remainder of the luminal anatomic structure intact. One example of this can be a covering or a stent, such as an intraluminal stent. Such a stent or covering can, for example, impart mechanical stability, act as a cover, or maintain at least partial patency of the structure it is covering (e.g. a luminal anatomic structure). The stent or covering may in certain examples be a resizable stent or covering that at least imparts mechanical stability, covers, or maintains at least partial patency of the anatomic structure. In this exemplary way, the stent or covering does not need to be fitted in diameter to be of a predetermined size, and overlapping areas of the membrane device take up the slack upon deployment of the device.

In another exemplary embodiments of the present invention, a number of different device patterns are described that enhance or enable different biological functions or capabilities.

The biological membrane may be conformed to be in a certain exemplary geometry. For example, the biological membrane may be conformed in a cylinder, a plane, any geometry preformed to the contour of the tissue of interest, or preformed to a desired contour to effect the best clinical treatment. In certain preferred examples, the cylinder is used as a stent or a covering.

In other examples, the edges of the biological membrane are tapered, and in certain preferred embodiments, may contain projections. The projections can comprise amniotic membrane, metal struts, nitinol struts, plastic struts, or composites, such as Polytetrafluoroethylene (PTFE), teflon, plastic, rubber, nitinol, or biodegradable composites or the like.

The biological membrane may be configured with holes. The biological membrane may have be configured to have 1, 2, 3, 5, 10, 20, 50, 100, 150, 200, 300, 500, or more holes, or different number of holes. The holes in the membrane can be of any geometry and may be configured to allow for passage of intraluminal tissues such as, but not limited to, blood, bile, or lymph to pass through. The exemplary minimum diameter of the holes may be between 10, 20, 30 40, 50, 75, 100, 200, 400, 500, 600, 750 μm in order to allow the passage of red and white blood cells, but other diameters are conceivable, and are within the scope of the present invention. The exemplary pattern of holes may be configured to allow endothelial or epithelial cells or other cells to migrate through the membrane device.

The holes and intervening spaces may be configured to impart further mechanical stability to the membrane device. For example, the edges of the membrane device may be tapered to further significantly improve endothelial or epithelial cell migration.

Accordingly, it is one object of the present invention that the exemplary membrane device that attaches to a luminal anatomic structure promotes re-endothelialization or re-epithelialization of said anatomic structure. Such exemplary membrane device thereby can be configured to allow the endothelial or epithelial cells of the luminal anatomic structure to migrate and cover the stent following deployment of the exemplary photoactivatable membrane (PAM) device. Promotion of this healing process can be facilitated by adjusting an exemplary PAM thickness, number and size of holes or openings, and by applying other pharmacological agents to the PAM device that facilitate said re-endo or re-epithelialization

The exemplary membrane device may be, in certain embodiments, comprised of layers of membrane, for example, amniotic membrane, configured to impart substantially more thickness and/or mechanical stability to the membrane device. The membranes of the exemplary embodiments of the present invention, e.g., photoactivatable membranes (PAM), may be modified to change shape or configuration. For example, the biological membranes can be comprised of layers of one or more, for example, 2, 3, 5, 10, 20, 30, 50 or more membrane sheets. These sheets can be affixed to each other, in certain examples, by electromagnetic radiation.

In one exemplary embodiment, the layers may be affixed to one another by means of applying electromagnetic radiation to layers of amniotic membrane comprised of photoactivatable dye.

### Exemplary Embodiments of Methods

The exemplary embodiments of the methods described herein can be suitable for use in a variety of applications, including in vitro laboratory applications, ex vivo tissue treatments, but especially in in vivo procedures on living subjects, e.g., humans, and especially in repairing luminal anatomical structures.

The exemplary methods described herein can be useful for adhering biological membranes to luminal anatomical structures using photosensitizing agents and electromagnetic energy. The exemplary methods described herein can also be useful for stabilizing luminal anatomical structures and for treating or preventing atherosclerotic plaques.

The exemplary methods described herein can be used, for example, for tissue bonding. Tissue bonding can be used to seal anatomical sites, for instance, after injury, or after a surgical procedure, or as part of a prophylactic measure to prevent against a disease or pathological event. In one example, for instance, an exemplary PAM tissue bonding technique/procedure has been previously used to seal neurorraphy sites [Photochemical Sealing Improves Outcome Following Peripheral Neurorrhaphy. A. C. O'Neill, M. A. Randolph, K. E. Bujold, I. E. Kochevar, R. W. Redmond, J. M. Winograd submitted to Experimental Neurology], incorporated by reference in its entirety herein. In this example, Rose Bengal-stained PAM was wrapped around the repair site (rat sciatic nerve) and exposed to 30 J/cm² (on each side) 532 nm (irradiance=0.5 W/cm²) using a frequency doubled Nd/YAG laser. For example, the PAM can additionally rapidly bond to vocal fold (epithelial, lamina propria and muscle layers) [unpublished], incorporated by reference in its entirety herein. In this example, bonding of PAM to cornea (without epithelial layer) an energy density of 100 J/cm² is typically used. PAM has also been bonded to dermis, epidermis and tracheal submucosa.

The exemplary embodiment of the instant invention describes exemplary methods for adhering a biological membrane to luminal anatomical structures. The exemplary method can comprise contacting a biological membrane with a photosensitizer agent and deploying the biological membrane photosensitizer complex to the luminal anatomical structure of interest, and then applying electromagnetic energy, thereby adhering the biological membrane to the luminal anatomical structure.

Another exemplary embodiment of the method according to the present invention can be provided for stabilizing a luminal anatomical structure. The exemplary method can comprise contacting a biological membrane with a photosensitizer agent and then deploying the biological membrane to the luminal anatomical structure in need of stabilization, applying electromagnetic energy to the biological membrane-photosensitizer complex in a manner effective to bond the tissue, and thereby stabilizing a luminal anatomical structure.

Still another exemplary embodiment of the present invention as described herein can provide an exemplary method for treating or preventing an atherosclerotic plaque. The exemplary method comprises identifying an atherosclerotic plaque, contacting a biological membrane with a photosensitizer agent, deploying the biological membrane to the atherosclerotic plaque, and applying electromagnetic energy to the biological membrane photosensitizer complex in a manner effective to bond the tissue, and thus treating or preventing an atherosclerotic plaque.

According to yet another exemplary embodiment of the present invention, exemplary methods for promoting one or more of cell growth and migration in a luminal anatomical structure of interest can be provided. The exemplary method can comprise contacting a biological membrane with a photosensitizer agent, deploying the biological membrane photosensitizer complex to the luminal anatomical structure of interest, and applying electromagnetic energy, and thereby promoting cell growth and migration in a luminal anatomical structure of interest.

### Exemplary Kits

The exemplary embodiments of the present invention can also provide kits for use in photochemical tissue bonding according to the exemplary methods as described herein. Such exemplary kits can be used for laboratory or for clinical applications. Such kits include PAM as described herein, a photosensitizer agent, e.g., a photosensitizer described herein, and instructions for adhering biological membrane to luminal anatomical structures, for stabilizing a luminal anatomical structure, for treating or preventing an atherosclerotic plaque, or for promoting one or more of cell growth and migration in a luminal anatomical structure of interest as described herein. The exemplary kits can include a container for storage, e.g., a light-protected and/or refrigerated container for storage of the photosensitizer agent. A photosensitizer included in the kits can be provided in various forms, e.g., in powdered, lyophilized, crystal, or liquid form.

The exemplary kits can include instructions for use.

### EXAMPLES

It should be appreciated that the exemplary embodiments of the present invention should not be construed to be limited to the examples that are now describes; rather, the exemplary embodiments of the present invention should be construed to include any and all applications provided herein and all variations within the skill of the ordinary artisan.

### Example 1

### Photoactivatable Biological Membrane (PAM) Device

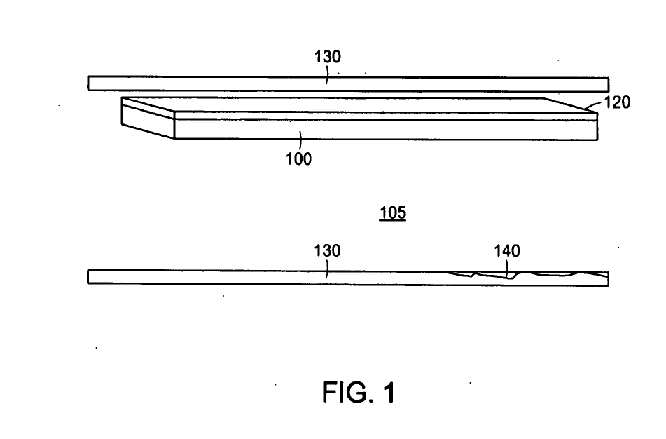

FIG. 1 depicts a small portion of an exemplary PAM device. The exemplary PAM device comprises a biological membrane 100, such as an amniotic membrane, which may be taken from the amnion of an animal, such as cow, pig, sheep, or the like, or alternatively in an exemplary embodiment from a human pregnancy, post-partum. The biologic membrane is constructed through synthetic chemistry means. In a preferred embodiment the biological membrane contains an epithelial surface, basement membrane, and connective tissue layers. The biological membrane, which in certain examples can preferably be an amniotic membrane, is coated or comprised there through of a photoactivatable dye 120. In certain preferred embodiments the dye is Rose Bengal. Alternatively, other photoactivatable dyes, including riboflavin-5-phosphate and methylene blue may be used. Photoactivatable dyes Rose Bengal, riboflavin-5-phosphate and methylene blue are typically activated using electromagnetic radiation centered at wavelengths of about 532 nm (Rose Bengal), 488 nm (riboflavin-5-phosphate) and 660 nm (methylene blue), respectively.

The exemplary PAM device can be placed within the lumen of the luminal anatomic structure 105. The side containing the substantially largest concentration of density of photoactivatable dye 120 can be placed immediately adjacent to, against, in close proximity to or in a close contact, or in certain examples within less than 1 μm from the luminal surface of the luminal anatomic structure 130. In one exemplary embodiment, the luminal surface of the luminal anatomic structure is kept substantially free of moisture or contaminants. In another exemplary embodiment, the connective tissue or basement membrane portion of the biological membrane can be placed immediately adjacent to, against or in substantially close proximity to the epithelial or endothelial surface of the luminal anatomic structure 130. In another example, the photoactivatable dye 120 may preferentially be located on the basement membrane or connective tissue portions of the biological membrane.

The luminal anatomic structure to which the exemplary PAM device is applied may be, but does not have to be, altered prior to application of the PAM device. In one exemplary embodiment, the luminal anatomic structure is not altered prior to application of the PAM device. In another example, the epithelial or endothelial cells of the luminal anatomic structure 130 are substantially removed prior to bonding the biological membrane to the luminal anatomic structure. In yet another exemplary embodiment, the luminal anatomic structure can be pretreated in some form including abrasion 140 by the exemplary PAM device deployment device to change the affinity characteristics of the PAM device for the luminal surface of the luminal anatomic structure.

### Example 2

### Configuring the Exemplary PAM Device

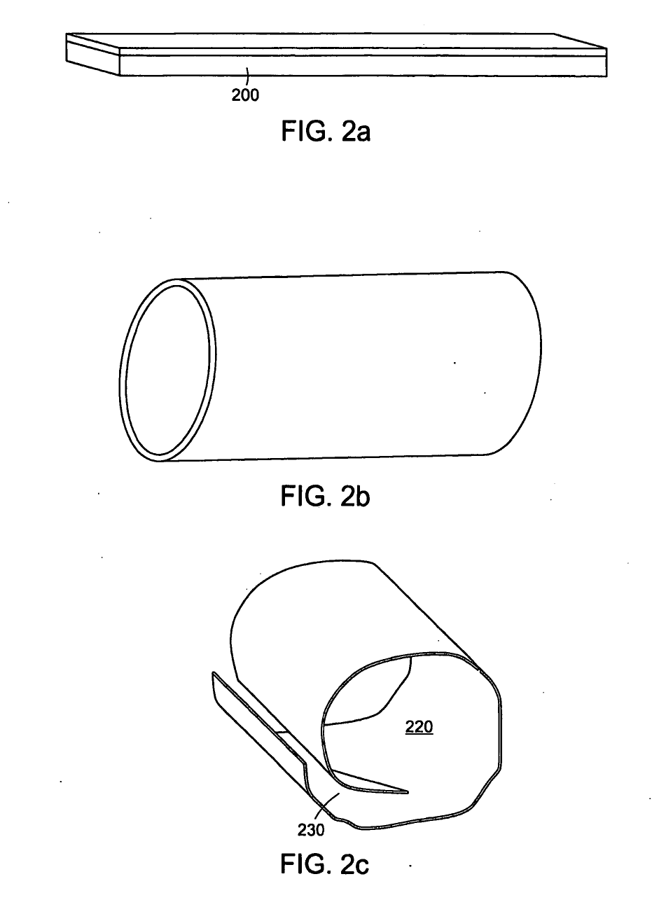

FIG. 2 is a diagram of different exemplary PAM device configurations. These exemplary configurations can comprise different topologies of the PAM device that are formed prior to deployment, during, or after deployment to conform to and/or alter the topology of the luminal anatomic structure. FIG. 2a depicts an exemplary PAM device configuration that comprises a sheet of PAM 200. This configuration in one exemplary embodiment can be appropriate for imparting stability to one portion of the luminal anatomical structure. In another embodiment this topology may be used to repair a defect in at least one portion of the luminal anatomic structure. In yet another exemplary embodiment, this configuration may be used to treat, repair, or cover only the portion of the luminal anatomic structure that requires alteration while leaving the remainder of the luminal anatomic structure intact. FIG. 2b depicts another exemplary PAM device configuration whereby the PAM device can be a circular cylinder 210. In one exemplary embodiment this configuration serves as an intraluminal stent or covering that at least imparts mechanical stability, covers, or maintains at least partial patency of said luminal anatomic structure. FIG. 2c shows a further exemplary PAM device configuration whereby the PAM device can be an overlapping circular cylinder 220. In this preferred embodiment, the PAM device may serve as a resizable stent or covering that at least imparts mechanical stability, covers, or maintains at least partial patency of said luminal anatomic structure. As opposed to FIG. 2b, this exemplary embodiment of FIG. 2c does not utilize the PAM device as being fitted in diameter to a predetermined knowledge of the diameter of the anatomic luminal structure. Overlapping areas 240 of the PAM device take up the slack upon deployment of the PAM device using the PAM deployment device.

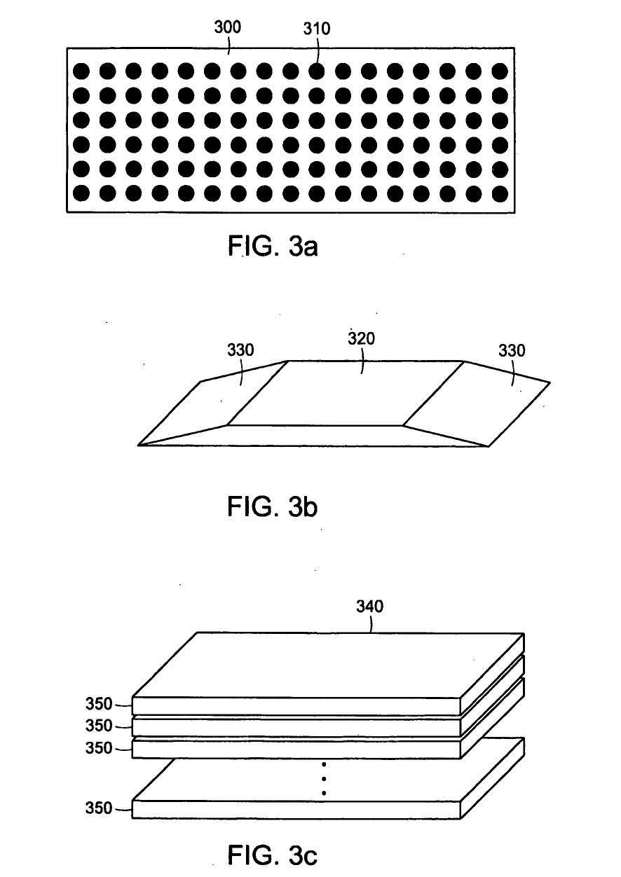

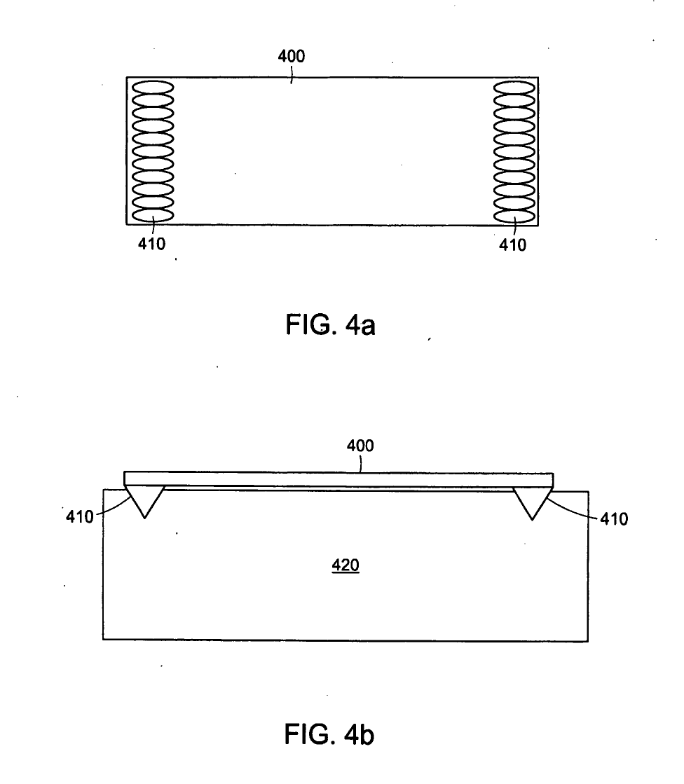

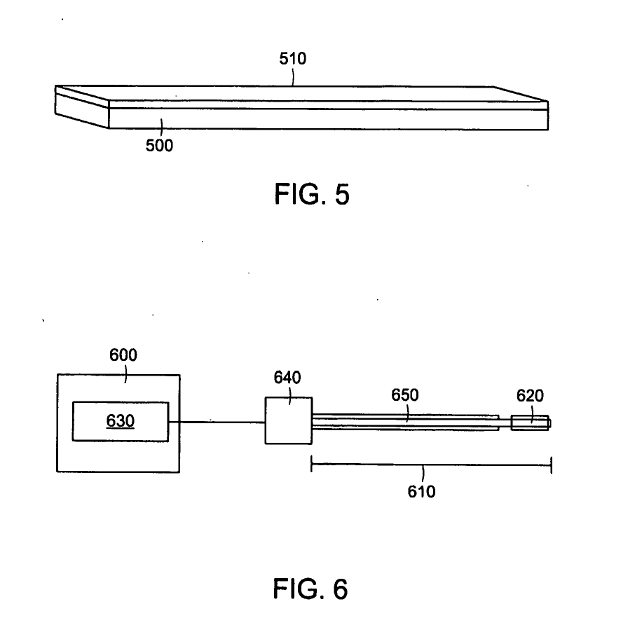

FIG. 3 depicts exemplary embodiments of PAM device patterns that can be configured to enhance or enable different biological functions or capabilities. FIG. 3a shows an exemplary PAM device 300 with holes 310 therein. The holes in this device can be of any geometry and are configured in one exemplary embodiment to allow for passage of intraluminal tissues such as blood, bile, or lymph to pass there through. In one further exemplary embodiment, the minimum diameter of such holes is between 10 and 500 um in order to allow the passage of red and white blood cells. In another exemplary embodiment, the pattern of holes is configured to substantially allow endothelial or epithelial cells or other cells to migrate through the PAM device therein. In yet another exemplary embodiment, the holes and intervening spaces can be further configured to impart further mechanical stability to the PAM device. FIG. 3b shows a cross-section of an exemplary embodiment of the PAM device 320 where the edges of the PAM device are tapered 330 to further significantly improve endothelial or epithelial cell migration. FIG. 3c depicts yet another exemplary embodiment of the PAM device 340 where the PAM device configuration is comprised of layers of amniotic membrane 350 configured to impart substantially more thickness and/or mechanical stability to the PAM device. In one exemplary embodiment, the layers may be affixed to one another by applying electromagnetic radiation to layers of amniotic membrane comprised of photoactivatable dye.

The mechanical characteristics of the exemplary PAM device may be altered by applying further electromagnetic radiation either before or after deployment within the luminal anatomic structure. Application of further electromagnetic radiation before placing the biological membrane on the PAM device deployment device, or after it has been placed in said deployment device can, in an exemplary embodiment, make the PAM device stiffer, thereby giving it mechanical properties for maintaining patency of a luminal anatomic structure. Alternatively, in another exemplary embodiment, further electromagnetic radiation may be applied after the PAM device has been bonded to the luminal anatomic structure, and, in a likewise manner, provide further mechanical stability to said PAM device. In yet another embodiment, further electromagnetic radiation may be applied to the PAM device both prior to and following the PAM device deployment in the luminal anatomic structure.

### Example 3

### Modifying the Luminal Surface for the PAM Device

One aspect of the exemplary PAM device is that when it is deployed, it may be prevented or limited to further substantially move within, across, or along the luminal surface of the luminal anatomic structure. In part, motion is prohibited by the affixation characteristics of the photoactivatable dye. Affixation characteristics of the PAM device

with respect to the luminal surface of the luminal anatomic structure may be further enhanced by a modification of the luminal surface. In one exemplary embodiment, this may be accomplished by at least one of partially scraping or abrading the epithelial or endothelial cell layer of the luminal surface of the luminal anatomic structure 140. In another exemplary embodiment, the way for enhancing the anchoring properties of the PAM device include further enhancing the PAM device so that the edges of said PAM device become further affixed to the anatomic luminal structure. In one exemplary embodiment, this further affixing way and/or arrangement can comprise patterning the PAM device 400 so that it may contain further projections 410 that are embedded into the luminal anatomic structure 420 during deployment. The further projections of the PAM device may include additional portions of amniotic membrane, metal or nitinol struts, or struts containing other plastics, composites or the like.

### Example 4

### Modification of the PAM Device-Coating and Additional Molecules

In addition to amniotic membrane and the photoactivatable dye of the PAM device 500, the exemplary PAM device may be coated or may comprise additional molecules 510 that convey properties to the PAM device preferable for achieving exemplary biological functions or characteristics following deployment. In one exemplary embodiment, the PAM device is coated or is comprised therein of anticoagulant molecules, such as, but not limited to glycoprotein IIb/IIIa inhibitors, aspirin or COX inhibitor analogs thereof, heparin, coumadin, or other biological molecules that inhibit the clotting cascade thereof. In another exemplary embodiment, the PAM device can be modified so that molecules that enhance endothelial or epithelial cell migration are incorporated into the surface or within said PAM device thereof Surface migration enhancement molecules include but are not limited to the integrins, selectins, cadherins, arginine-glycine-aspartate (RGD) peptides, and albumin. Additionally chemotaxis molecules may be incorporated into the PAM device therein. In another exemplary embodiment, the molecules designed to prohibit an overly aggressive or vigorous healing response may be added, including, but not limited to immunosuppressive agents such as rapamycin, tobramycin, or the like, antiproliferative agents, such as paclitaxel or the like, chemotherapeutic agents such as 5-fluorouracil, or the like and HMG CoA reductase inhibitors, or the like. In yet another preferred embodiment, the PAM device is configured to additionally contain molecules that substantially cause the healing process to occur, including TGF-b, platelet derived growth factor PDGF, fibroblast growth factor (FGF) or the like. These and other molecules described thereof may be attached to the PAM device by surface modification of the PAM device and chemical conjugation of said molecules to the surface modifications of said PAM device. Alternatively, antibodies to the molecules may be attached to the surface modifications by covalent bonding. In still another exemplary embodiment, a photochemical reaction can be applied to the exemplary PAM device in the presence of said molecules whereby the said molecules become incorporated in the PAM device.

### Example 5

### Exemplary PAM System

FIG. 6 depicts a schematic of an exemplary embodiment of the PAM system, that incorporates a PAM system console 600, a PAM device deployment device 610 comprising a probe such as a catheter or endoscope or the like, and the PAM device itself 620. In one exemplary embodiment, the PAM system comprises a source of electromagnetic radiation 630 that is associated with the PAM device deployment device. The source of electromagnetic radiation 630 can be controlled by the operator so that when the PAM device is placed in or near close approximation to the luminal surface of the luminal anatomic structure, the electromagnetic radiation is enabled so that the PAM device is exposed to electromagnetic radiation that activates said photoactivatable dye. In another exemplary embodiment, the electromagnetic radiation is coupled to the PAM device deployment device through a coupling junction 640 to an optical fiber apparatus 650 that resides within the PAM device deployment device 610. In one further exemplary embodiment, the electromagnetic radiation coupling junction 640 is comprised of an optical connector. The optical fiber apparatus 650 conveys electromagnetic radiation to the distal end of 610 and terminates in a manner such that 620 is irradiated by the electromagnetic radiation.

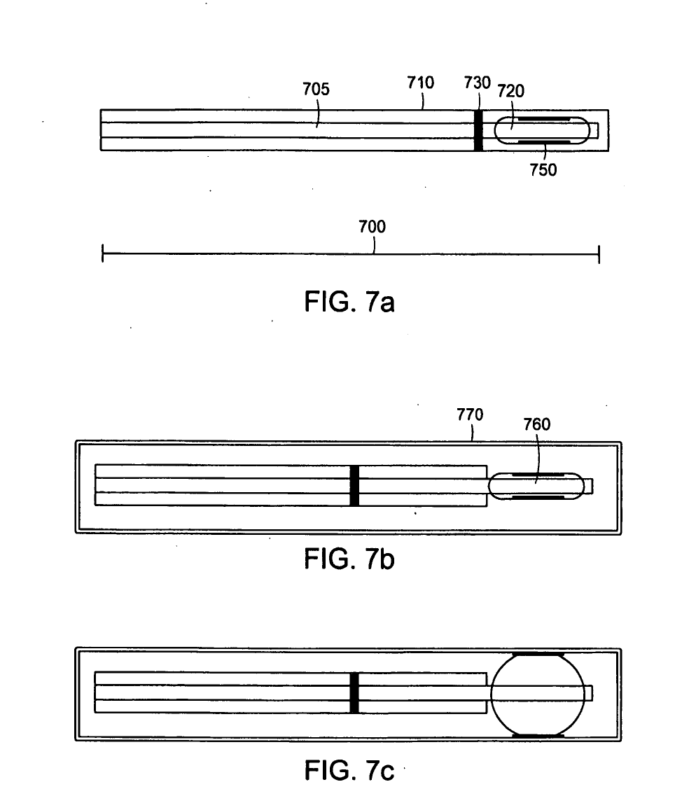

FIG. 7 depicts an expanded view of an exemplary embodiment of the distal portion of the PAM device deployment device 700. In this exemplary embodiment, the optical fiber apparatus 705 can be enclosed with a sheath 710. The sheath can additionally house a balloon deployment device 720 that is contained within 710 while 700 is advanced to the region of interest within the luminal anatomical structure. The balloon of the balloon deployment device may be configured to be associated with the exemplary PAM device such that when the balloon is inflated, the PAM device is situated substantially near to the luminal surface of the luminal anatomic structure. In one preferred embodiment this localization of the balloon deployment device is accomplished by use of radioopaque markers 730 that reside on or within the sheath 710. When the balloon deployment device is in a position of interest that is predetermined, the outer sheath 710 may be retracted to expose the balloon deployment device 760 within the lumen of the luminal anatomic structure 770. In yet a further exemplary embodiment, the PAM device deployment device can be a catheter configured for insertion into a coronary artery following percutaneous transluminal route. The catheter may comprise at least one of a provision for saline flushing, insertion over the guide wire provision, or a rapid exchange guide wire provision.

The exemplary PAM device may be mounted on the balloon deployment device in a manner such that when the balloon is deflated, the PAM device is placed within close proximity to the luminal surface of the luminal anatomic structure (e.g., see FIG. 7c). In another exemplary embodiment, the balloon of the balloon deployment device can be a soft balloon with compliant and or elastic mechanical characteristics and can be deflated with low pressure. The balloon may be inflated by use of air or saline injected manually or automatically. In another exemplary embodiment, a pressure sensor, optical sensor, or other sensor can be incorporated in the PAM device deployment device to sense when the balloon deployment device is located at the region of interest or to sense when the PAM device attached to the balloon is in close proximity to the luminal surface of the luminal anatomic structure. The balloon deployment device may then be inflated to place the PAM device in close proximity to the luminal surface of the luminal anatomic structure.

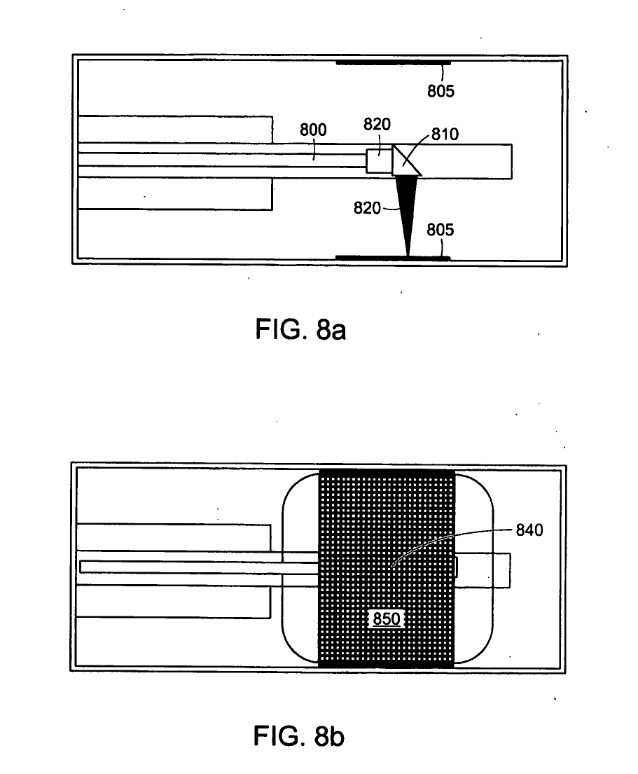

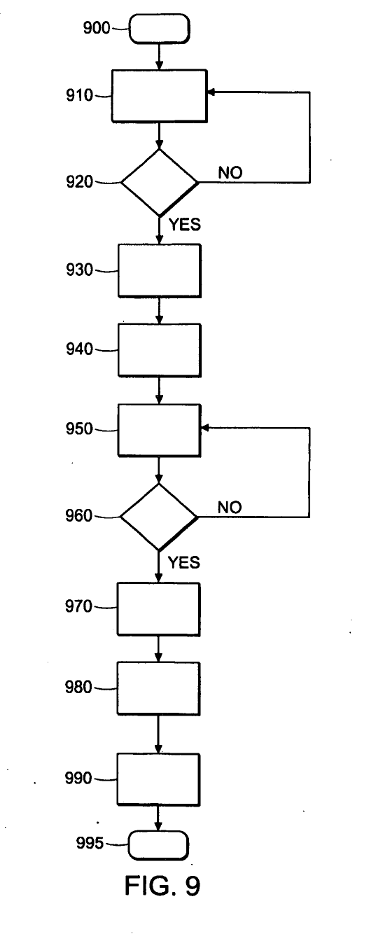

FIG. 8 depicts schematic diagrams of different exemplary embodiments of distal optics present in the exemplary PAM device deployment device. The exemplary device can be operatively configured to transmit radiation from the optical fiber configuration 800 through the balloon deployment device and onto the PAM device itself. The optical configuration may contain a waveguide or plurality of waveguides preferably one or more optical fibers that resides within the sheath and is configured to transmit electromagnetic radiation from the proximal to distal end of the probe and onto the PAM device 805. The same waveguide or plurality of waveguides or a further optical fiber configuration may receive light remitted from the PAM device. The electromagnetic radiation can be directed to the PAM device by distal optical components 810, 820 that comprise at least one of a deflector (prism, angled fiber face, diffraction grating or the like) 810 and may further comprise a lens apparatus 820 for focusing said electromagnetic radiation onto at least one portion of the PAM device. In one embodiment, as shown, e.g., in FIG. 8a, the radiation 830 may be directed to at least one specific region of the PAM device, whereas in another embodiment (FIG. 8b) the electromagnetic radiation 850 is configured to diffuse broadly and cover a substantial portion of the PAM device. The diffusing arrangement according to the exemplary embodiment may include a diffusing apparatus 840 such as a diffuser, axicon, or other diffusing optic for diffusing the electromagnetic radiation over a substantial portion of the PAM device and the luminal surface of the luminal anatomic structure. In another exemplary embodiment, a further optical sensing apparatus can be provided that may be used to detect photobleaching and determine when the photoactivation is complete by transceiving at least one of reflected, absorbed, or emitted light from the PAM device.

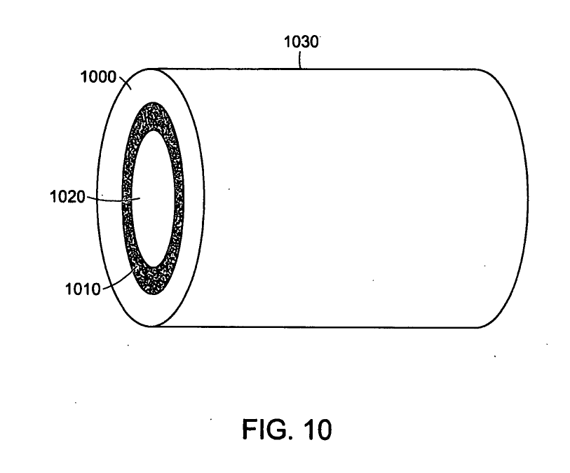

When the lumen of the luminal anatomic structure is stenotic, blocked, or otherwise at least partially closed and at least one purpose of the intraluminal device is to maintain patency of the luminal anatomic structure, it may be additionally advantageous to configure the PAM device so that it is associated with or incorporates another mechanical device, such as a conventional bare metal or other drug-eluting stent. FIG. 10 depicts an exemplary embodiment, comprising a PAM device 1010 that is coupled to a stent 1000. The PAM device may contain the stent or be physically coupled or associated with a stent. In another embodiment, the PAM device may only be coupled or associated with the stent for portions of the stent. The PAM device may be associated with the stent on at least one of the luminal side of the stent 1020 or the tissue side of the stent 1030. The PAM device may be placed on the luminal side of the stent to promote reepi- or reendothelialization. The PAM device may contain holes that to facilitate endothelialization. In yet another embodiment, the holes may correspond to the stent pattern. The PAM device and stent may be deployed utilized the same balloon expansion mechanism simultaneously or serially; for instance in one embodiment, the stent is deployed and the PAM device is subsequently deployed, in another embodiment, the PAM device is deployed and the stent is subsequently deployed. As in other embodiments, following deployment of the PAM device, electromagnetic radiation is applied to the PAM device to affix the PAM device to the luminal aspect of the luminal anatomic structure. In yet a further embodiment of this invention, the stent affixes the PAM device to the luminal anatomic structure, and no electromagnetic radiation is applied.

### Example 6

### Deploying the Exemplary PAM Device

FIG. 9 depicts a flow chart describing an exemplary embodiment of a method for deploying the exemplary PAM device in a coronary artery. Upon starting the procedure 900, the catheter device can be positioned within the target location within the artery in step 910. In an exemplary embodiment, the catheter device may be placed over a guide wire using standard percutaneous procedures. The it is determined whether an angiographic localization of the radioopaque marker is in target lesion query in step 920. If the answer is NO, repositioning is repeated. If the answer is YES, a sheath can be retracted, exposing the balloon deployment device to the lumen of the artery in step 930. The balloon may be inflated in step 940, likely placing the PAM device in substantially close proximity to the luminal surface of the coronary artery. Electromagnetic radiation can then be applied to the PAM device and the artery in step 950. Feedback from the receiving optical arrangement may be used to determine when the PAM device is substantially activated in step 960. If no such substantial activation occurred, the PAM device can continue to be exposed to the electromagnetic radiation. Otherwise, the exemplary procedure is complete in step 970, and the balloon may be deflated in step 980. The sheath can be protracted to cover the balloon in step 990. The exemplary PAM device deployment device may then be withdrawn from artery, and the procedure can be completed in step 995.

### Example 7

### Exemplary Bonding a Biological Membrane to an Endothelial Surface

According to another exemplary embodiment, a biological membrane can be bonded to the endothelial surface of an excised artery. A freshly harvested swine carotid artery may be opened longitudinally and cut into, e.g., 6-8 mm lengths. Of course other lengths can be used. The artery lengths may be placed on a paper backing to maintain a flat surface. The surface can be blotted dry. The connective tissue surface of a piece of human amniotic membrane, in certain examples, a ˜4×4 mm piece of human amniotic membrane (on nitrocellulose paper backing), may be stained with a photosensitizer agent. In certain exemplary embodiments, the photosensitizer agent of Rose Bengal (RB) can be used, however one of skill in the art understands that the exemplary embodiments of the present invention is not limited to the photosensitizer agent being only RB. Other photosensitizers can include but not limited to, for example, Rose Bengal (RB), riboflavin-5-phosphate (R-5-P), methylene blue (MB), or N-hydroxypyridine-2-(1H)-thione (N-HTP). Preferably, staining is carried out with 0.1% Rose Bengal (in PBS) for 1 min and then excess dye is blotted. The stained surface of PAM can then be placed in contact with the luminal surface of the artery and exposed to a source of electromagnetic energy. In other exemplary embodiments, the electromagnetic energy can be, e.g., 30 J/cm² 532 nm (frequency-doubled Nd/YAG). This may result in bonding of the PAM to the endothelial surface with good bond strength.

### Incorporation by Reference

The contents of all references, patents, pending patent applications and published patents, cited throughout this application are hereby expressly incorporated by reference. Equivalents

Those skilled in the art will recognize, or be able to ascertain using no more than routine experimentation, many equivalents to the specific embodiments of the invention described herein. Such equivalents are intended to be encompassed by the following claims.

1. A method for adhering a biological membrane to a luminal anatomical structure, the method comprising:
contacting a biological membrane with a photosensitizer agent;
deploying the biological membrane-photosensitizer complex to the luminal surface of the luminal anatomical structure of interest; and
applying electromagnetic energy; thereby adhering the biological membrane to the luminal anatomical structure.

2. A method for altering the mechanical characteristics of a luminal anatomical structure, the method comprising:
contacting a biological membrane with a photosensitizer agent;
deploying the biological membrane to the luminal anatomical structure in need of stabilization;
applying electromagnetic energy to the biological membrane-photosensitizer complex in a manner effective to bond the tissue; thereby altering the mechanical characteristics of the luminal anatomical structure.

3. A method for treating or preventing an atherosclerotic plaque, the method comprising:
contacting a biological membrane with a photosensitizer agent;
deploying the biological membrane to the atherosclerotic plaque;
applying electromagnetic energy to the biological membrane-photosensitizer complex in a manner effective to bond the membrane-photosensitizer to the plaque;
thereby treating or preventing an atherosclerotic plaque.

4. (canceled)

5. (canceled)

6. The method of claim 1, wherein the electromagnetic energy is applied to the one or both of the biological membrane and the luminal anatomical structure.

7. The method of claim 6, wherein the electromagnetic energy is applied to the one or both of the biological membrane and the luminal anatomical structure in a manner effective to bond the tissue.

8. The method of claim 7, wherein the electromagnetic energy is electromagnetic radiation.

9. The method of claim 7, wherein the electromagnetic radiation is at a wavelength between about 532 nm and 660 nm.

10. (canceled)

11. The method of claim 1, wherein the contacting step occurs ex vivo or in vivo in a subject.

12. (canceled)

13. A photoactivatable membrane device comprising a biological membrane and a photosensitizer agent.

14. The method of claim 1, wherein the biological membrane is an amniotic membrane.

15. The method of claim 14, wherein the biological membrane is from a mammal or is synthetic.

16. The method of claim 15, wherein the mammal is a human.

17. (canceled)

18. The method of claim 1, wherein the biological membrane contains an epithelial layer, a basement membrane layer, and a connective tissue layer.

19. The method of claim 18, wherein the photosensitizer agent is coated on the biological membrane.

20. The method of claim 19, wherein the photosensitizer agent is coated on one or more of the epithelial layer, the basement membrane layer or the connective tissue layer of the biological membrane.

21. The method of claim 19, wherein the photosensitizer agent is coated on the biological membrane in a gradient from highest concentration to lowest concentration.

22. The method of claim 21, wherein the side of the biological membrane with the highest concentration of photosensitizer agent is placed 1 μm or less from a luminal surface of a luminal anatomic structure.

23. The method of claim 1, wherein the biological membrane is coated with additional biological molecules.

24. The method of claim 23, wherein the additional biological molecules are selected from the group consisting of at least one of: anticoagulants, molecules that enhance cell migration, proteases, molecules that enhance chemotaxis, chemoattractants, immunosuppressive agents, chemotherapeutic agents, molecules that enhance cell growth, molecules that enhance healing, antibodies, small molecule inhibitors, and anti-inflammatories, antioxidants.

25. The method of claim 24, wherein the antibodies are attached to the biological membrane covalently.

26. The method of claim 24, wherein the additional biological molecules are attached to the biological membrane by applying a second dose of electromagnetic energy.

27. The method of claim 1, wherein the photosensitizer agent is a compound selected from the group consisting of at least one of: xanthenes, flavins, thiazines, porphyrins, expanded porphyrins, and chlorophylis.

28-30. (canceled)

31. A method for promoting cell growth in a luminal anatomical structure of interest, the method comprising:
contacting a biological membrane with a photosensitizer agent;
deploying the biological membrane-photosensitizer complex to the luminal anatomical structure of interest; and
applying electromagnetic energy; thereby promoting cell growth and migration in a luminal anatomical structure of interest.

32-53. (canceled)

54. A kit comprising a biological membrane and a photosensitizer agent for use in the methods of claim 1, and instructions for use.

55. An arrangement for coupling to a luminal anatomical structure, comprising:
a particular structure having a form so as to be transportable within the luminal anatomical structure, wherein the structure includes a photo-activatable substance which, when activated by at least one electro-magnetic radiation, couples to at least one portion of the luminal anatomical structure.

56-74. (canceled)
